(19) United States
(12) Patent Application Publication
(10) Pub. No.: US 2026/0306202 A1
(43) Pub. Date: Oct. 8, 2026

(54) ASSISTED POSITIONING OF A REPOSITIONABLE STRUCTURE

(71) Applicant: INTUITIVE SURGICAL OPERATIONS, INC., Sunnyvale, CA (US)

(72) Inventors: Pavel CHTCHEPROV, Durham, NC (US); Paul W. MOHR, Mountain View, CA (US); Daniel W. NISSENBAUM, San Francisco, CA (US); Trevor PIER, Redwood City, CA (US); Prasad V. UPADRASTA, San Jose, CA (US); Zhuoqun XU, Sunnyvale, CA (US)

(21) Appl. No.: 19/478,523

(22) PCT Filed: Apr. 25, 2024
(86) PCT No.: PCT/US2024/026338

§ 371 Date: Oct. 24, 2025

Related U.S. Application Data
(60) Provisional application No. 63/498,466, filed on Apr. 26, 2023.

(51) Int. Cl. A61B 34/37; A61B 50/13; G16H 20/40

(52) U.S. Cl. CPC A61B 34/37; A61B 50/13; G16H 20/40

(57) ABSTRACT

A system includes a base, a proximal repositionable structure supported by the base and a distal repositionable structure having a proximal portion supported by the proximal repositionable structure and a distal portion. An actuator system is configured to move the base, the proximal repositionable structure, and the distal repositionable structure. A control system is coupled to the actuator system and is configured to facilitate external repositioning of the distal portion that moves a control feature relative to the base frame, detect first repositioning and based on a first geometric parameter characterizing the control feature after the first repositioning, determine a first base movement of the base and a first structure movement of the proximal repositionable structure, wherein performing the first base movement and the first structure movement together moves a reference feature relative to the control feature, the reference feature fixed relative to a portion of the proximal repositionable structure.

### RELATED APPLICATIONS

This application claims the benefit to U.S. Provisional Application No. 63/498,466, filed Apr. 26, 2023, and entitled “Assisted Positioning of a Repositionable Structure,” the subject matter of which is incorporated by reference herein.

### TECHNICAL FIELD

The present disclosure relates generally to computer-assisted systems and more particularly relates to assisted positioning of a repositionable structure.

### BACKGROUND

Computer-assisted electronic systems are being used more and more often. This is especially true in industrial, entertainment, educational, and other settings. As a medical example, the medical facilities of today have large arrays of electronic systems being found in operating rooms, interventional suites, intensive care wards, emergency rooms, and/or the like. Some of these electronic systems are capable of autonomous or semi-autonomous motion or teleoperated motion. For example, a computer-assisted system, such as a robotic or other system can be used to perform a task at a worksite by controlling one or more repositionable structures of the computer-assisted system that are used to manipulate an instrument or other tool within the worksite. Many of these computer-assisted systems can also be physically manipulated into different configurations by external forces.

Oftentimes, the one or more repositionable structures or some other part(s) of the computer-assisted system have to be positioned appropriately within a physical environment in order to support movement of the instrument or tool within the worksite. As a medical example, the worksite can include an interior anatomy of a patient and the computer-assisted system and/or the one or more repositionable structures are positioned to support manipulation of the anatomy of the patient using a medical instrument. However, the physical environment around the manipulating device can include obstacles, such as other equipment, fixtures such as lighting fixtures, personnel, the patient in a medical example, and/or the like, that should be avoided when positioning the computer-assisted system.

Accordingly, improved techniques for assisting in the positioning or computer-assisted systems are desirable.

### SUMMARY

In some embodiments, a computer-assisted system includes a base having a base frame, a proximal repositionable structure supported by the base, a distal repositionable structure having a proximal portion and a distal portion. The proximal portion is supported by the proximal repositionable structure and an actuator system is configured to move the base, the proximal repositionable structure, and the distal repositionable structure. A control system is communicatively coupled to the actuator system and is configured to, in response to receiving a first indication to enter a first mode while not receiving a second indication not to enter the first mode, enter the first mode. In the first mode, the control system is further configured to facilitate external repositioning of the distal portion that moves a control feature relative to the base frame, wherein the control feature is fixed relative to the distal portion. In the first mode, the control system is further configured to detect a first repositioning of the distal portion that moves the control feature relative to the base frame. In the first mode, the control system is further configured to detect the first repositioning and based on a first geometric parameter characterizing the control feature after the first repositioning, a first base movement of the base relative to a world frame and a first structure movement of the proximal repositionable structure relative to the base frame, wherein performing the first base movement and the first structure movement together moves a reference feature relative to the control feature, and wherein the reference feature is fixed relative to a portion of the proximal repositionable structure. The control system commands the actuator system to move the computer-assisted system based on the first base movement and the first structure movement.

In some embodiments method includes receiving, by a control system, a first indication to enter a first mode with respect to a system, the computer assisted system. The computer assisted system includes a base having a base frame, a proximal repositionable structure supported by the base, a distal repositionable structure having a proximal portion and a distal portion. The proximal portion is supported by the proximal repositionable structure. The computer assisted system includes an actuator system configured to move the base, the proximal repositionable structure, and the distal repositionable structure, the control system being communicatively coupled to the actuator system. The method further includes, in response to receiving the first indication and failing to receive a second indication not to enter the first mode, entering, by the control system, the first mode. While the control system is in the first mode, the method includes facilitating, by the control system, external repositioning of the distal portion that moves a control feature relative to the base frame, wherein the control feature is fixed relative to the distal portion. The method includes detecting, by the control system, a first repositioning of the distal portion that moves the control feature relative to the base frame. The method includes determining, by the control system, in response to detecting the first repositioning and based on a first geometric parameter characterizing the control feature after the first repositioning, a first base movement of the base relative to a world frame and a first structure movement of the proximal repositionable structure relative to the base frame, wherein performing the first base movement and the first structure movement together moves a reference feature relative to the control feature, and wherein the reference feature is fixed relative to a portion of the proximal repositionable structure. The method includes commanding, by the control system, the actuator system to move the computer-assisted system based on the first base movement and the first structure movement.

In some embodiments a non-transitory machine-readable medium comprises a plurality of machine-readable instructions which when executed by one or more processors associated with a computer-assisted device are adapted to cause the one or more processors to perform the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 is a simplified diagram including an example computer-assisted system, according to various embodiments;

FIG. 2 depicts an example computer-assisted system with an illustrative configuration of a sensor system, according to various embodiments;

FIG. 3 is a simplified diagram showing an example computer-assisted system according to some embodiments;

FIG. 4 illustrates the control module of FIG. 1 in greater detail, according to various embodiments;

FIG. 5 is a flow diagram of example method steps for entering a positioning mode for a manipulating device, according to various embodiments;

FIG. 6 is a flow diagram of example method steps for implementing a first positioning mode for a manipulating device, according to various embodiments;

FIG. 7 is a flow diagram of example method steps for implementing the first positioning mode for the manipulating device while accounting for range of motion limits of the manipulating device, according to other various embodiments;

FIG. 8 is a flow diagram of example method steps for implementing a second positioning mode, according to various embodiments; and

FIG. 9 is a flow diagram of example method steps for selecting between positioning, or positioning and orienting, in the first or second repositioning modes, according to various embodiments.

### DETAILED DESCRIPTION

This description and the accompanying drawings that illustrate inventive aspects, embodiments, embodiments, or modules should not be taken as limiting-the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various elements (such as systems and devices, and portions of systems and devices) with examples in three-dimensional space. In such examples, the term “position” refers to the location of an element or a portion of an element in space, such as a three-dimensional space (e.g., three degrees of translational freedom along Cartesian x-, y-, and z-coordinates). Also in such examples, the term “orientation” refers to the rotational placement of an element or a portion of an element in space, such as a three-dimensional space (e.g., three degrees of rotational freedom—e.g., roll, pitch, and yaw). Other examples may encompass other dimensional spaces, such as two-dimensional spaces. As used herein, the term “pose” refers to the position, the orientation, or the position and the orientation combined, of an element or a portion of an element. As used herein, and for an element or portion of an element of a structure or assembly (e.g., of a computer-assisted system or a repositionable structure, etc.), the term “proximal” in a kinematic series refers to a direction toward the base of the kinematic series, and the term “distal” refers to a direction away from the base along the kinematic series.

Aspects of this disclosure are described in reference to electronic, computer-assisted, and robotic systems or devices, which may include systems and devices that are teleoperated, autonomous, semiautonomous, manually manipulated, and/or the like. Example computer-assisted systems include those that comprise robots or robotic devices. Further, aspects of this disclosure are described in terms of an embodiment using a medical system, such as the da Vinci@ Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

Computer-assisted systems can include one or more repositionable structures configured to manipulate one or more instruments supported by one of the repositionable structures. To aid setup of the instrument or the repositionable structure that supports and manipulates the instrument within a worksite, it is advantageous to configure one or more portions of the repositionable structure before the procedure. This can be done in a multitude of ways, such as based on an anticipated position and/or orientation of the instrument that will be used during the procedure, or through another technique.

As one example for setting up a computer-assisted system that is used for a medical procedure involving sterile and nonsterile areas, such as a procedure performed on the anatomy of a patient, configuration of the computer-assisted system including the one or more repositionable structures is performed by first and second operators working collaboratively. A first operator is located on a non-sterile side (e.g., a non-patient side) of the computer-assisted system. The first operator uses one or more non-sterile controls to position a moveable base of the repositionable structure to a location relative to the patient so that the computer-assisted system is appropriately positioned for manipulating one or more instruments during the procedure. To help guide the first operator, a second operator located on the sterile side of the computer-assisted system provides visual or verbal guidance to the first operator about how to move the base. In this example, optimal configuration of the computer-assisted system typically involves collaboration and communications between multiple persons. This setup process can be complex, lengthy, inefficient, and/or result in a less than optimal configuration of the computer-assisted system relative to the patient.

As is described in further detail below, an improved approach allows for an operator on one side of the repositionable structure (e.g., the sterile side, the patient side (which may not need to be sterile-depending on the procedure) to move the base of the repositionable structure at least some of the time. In an example, the operator first moves (e.g., manually, with or without assistive forces or torques from the computer-assisted system) a distal portion of the one or more repositionable structures to which an instrument will eventually be mounted to perform a procedure. The computer-assisted system monitors the position of a distal portion of the one or more repositionable structures. Based on the position of the one or more repositionable structures, the computer-assisted system controls the moveable base and/or the one or more repositionable structures in order to place the computer-assisted system and the one or more repositionable structures at or near a position that is determined based on the position of the distal portion. In this way, the single operator can manually move the distal portion toward a location close to the patient where a procedure is to be performed and/or an instrument is to be inserted into a patient. Additionally, the computer-assisted system can also monitor an orientation associated with the distal portion and control the moveable base and/or the one or more repositionable structures based on the orientation.

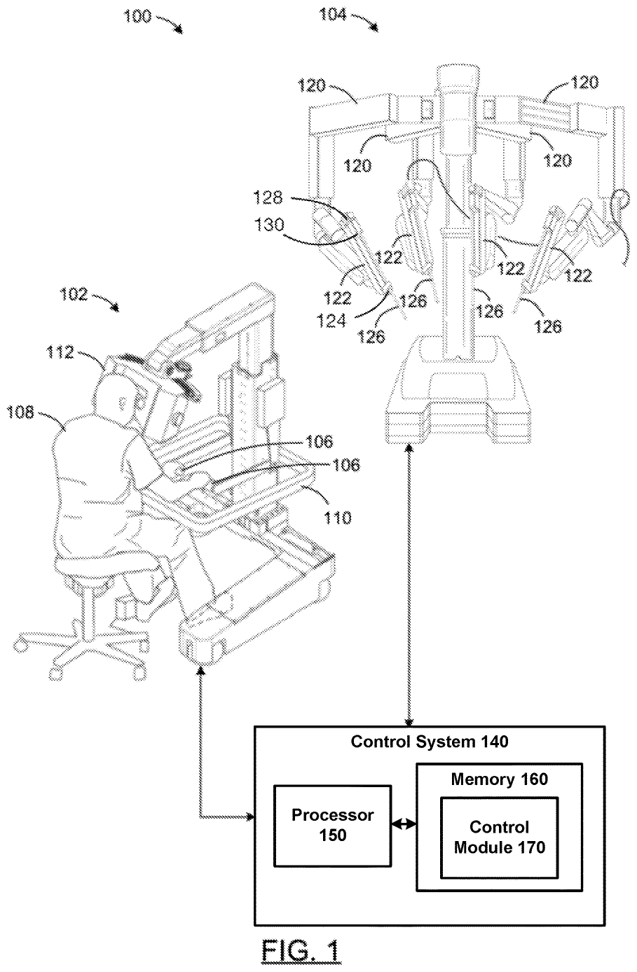

FIG. 1 is a simplified diagram of an example computer-assisted system 100, according to various embodiments. In some examples, the computer-assisted system 100 is a teleoperated system. In medical examples, computer-assisted system 100 can be a teleoperated medical system such as a surgical system. As shown, computer-assisted system 100 includes a follower device 104 that can be teleoperated by being controlled by one or more leader devices (also called “leader input devices” when designed to accept external input), described in greater detail below. In this example, the follower device 104 is a manipulating device comprising multiple repositionable structures. Systems that include a leader device and a follower device are referred to as leader-follower systems, and also sometimes referred to as master-slave systems. Although this example shows a leader-follower system, other computer-assisted system may not be leader-follower systems, and may be autonomous, semi-autonomous, or use a non-teleoperation-type of operator control. Also shown in FIG. 1 is an input system that includes a workstation 102 (e.g., a console), and in various embodiments the input system can be in any appropriate form and may or may not include a workstation 102.

In the example of FIG. 1, workstation 102 includes one or more leader input devices 106 that are designed to be contacted and manipulated by an operator 108. For example, workstation 102 can comprise one or more leader input devices 106 for use by the hands, the head, or some other body part(s) of operator 108. Leader input devices 106 in this example are supported by workstation 102 and can be mechanically grounded. In some embodiments, an ergonomic support 110 (e.g., forearm rest) can be provided on which operator 108 can rest his or her forearms. In some examples, operator 108 can perform tasks at a worksite near follower device 104 during a procedure by commanding follower device 104 using leader input devices 106.

A display unit 112 is also included in workstation 102. Display unit 112 can display images for viewing by operator 108. Display unit 112 can be moved in various degrees of freedom to accommodate the viewing position of operator 108 and/or to optionally provide control functions as another leader input device. In the example of computer-assisted system 100, displayed images can depict a worksite at which operator 108 is performing various tasks by manipulating leader input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

When using workstation 102, operator 108 can sit in a chair or other support in front of workstation 102, position his or her eyes in front of display unit 112, manipulate leader input devices 106, and rest his or her forearms on ergonomic support 110 as desired. In some embodiments, operator 108 can stand at the workstation or assume other poses, and display unit 112 and leader input devices 106 can be adjusted in position (height, depth, etc.) to accommodate operator 108.

In some embodiments, the one or more leader input devices 106 can be ungrounded. “Ungrounded” leader input devices are not kinematically grounded. Examples include leader input devices held by the hands of operator 108 without additional physical support. Such ungrounded leader input devices can be used in conjunction with display unit 112. In some embodiments, operator 108 can use a display unit 112 positioned near the worksite, such that operator 108 manually operates instruments at the worksite, such as a laparoscopic instrument in a surgical example, while viewing images displayed by display unit 112.

Computer-assisted system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can support, for example, an instrument 126. As shown, each instrument assembly 122 is mounted to a distal portion of a respective manipulator arm 120. The distal portion of each manipulator arm 120 further includes a cannula mount 124 which is configured to have a cannula (not shown) mounted thereto. When a cannula is mounted to the cannula mount, a shaft of an instrument 126 passes through the cannula and into a worksite, such as a surgery site during a surgical procedure. A cannula can include additional features, such as inflatable bags for expanding working space or providing pressurization, etc. A force transmission mechanism 130 of the instrument assembly 122 can be connected to an actuation interface assembly 128 of the manipulator arm 120 that includes drive and/or other mechanisms controllable from workstation 102 to transmit forces to the force transmission mechanism 130 to actuate the instrument 126.

In various embodiments, one or more of instruments 126 can include an imaging device for capturing images (e.g., optical cameras, hyperspectral cameras, ultrasonic sensors, etc.). For example, one or more of instruments 126 can be an endoscope assembly that includes an imaging device, which can provide captured images of a portion of the worksite to be displayed via display unit 112.

In some embodiments, the manipulator arms 120 and/or instrument assemblies 122 can be controlled to move and articulate instruments 126 in response to manipulation of leader input devices 106 by operator 108, and in this way “follow” the leader input devices 106 through teleoperation. This enables the operator 108 to perform tasks at the worksite using the manipulator arms 120 and/or instrument assemblies 122. Manipulator arms 120 are examples of repositionable structures that a manipulating device (e.g., follower device 104) can include. In some embodiments, a repositionable structure of a manipulating device can include a plurality of links that are rigid members and joints that are movable components that can be actuated to cause relative motion between adjacent links. For a surgical example, the operator 108 can direct follower manipulator arms 120 to move instruments 126 to perform surgical procedures at internal surgical sites through minimally invasive apertures or natural orifices.

As shown, a control system 140 is provided external to workstation 102 and communicates with workstation 102. The control system 140 is also called the “processing system 140” herein. In other embodiments, control system 140 can be provided in workstation 102 or in follower device 104. As operator 108 moves leader input device(s) 106, sensed spatial information including sensed position and/or orientation information is provided to control system 140 based on the movement of leader input devices 106. Control system 140 can determine or provide control signals to follower device 104 to control the movement of manipulator arms 120, instrument assemblies 122, and/or instruments 126 based on the received information and operator input. In one embodiment, control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like).

Control system 140 can be implemented on one or more computing systems. One or more computing systems can be used to control follower device 104. In addition, one or more computing systems can be used to control components of workstation 102, such as movement of a display unit 112.

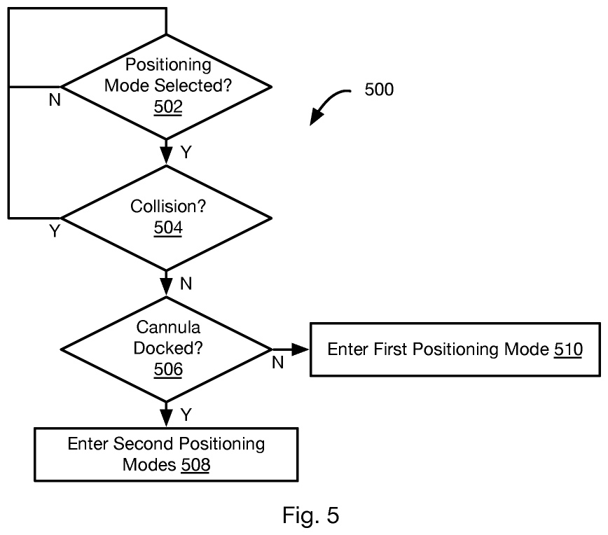

As shown, control system 140 includes a processor 150 and a memory 160 storing a control module 170. Although referred to in the singular herein, processor 150 can comprise one or more processors in various instances. In addition, processor 150 can further include supporting circuitry such as power conditioning circuitry. Operation of the control system 140 is controlled by processor 150. Control system 140 can be implemented as a stand-alone subsystem and/or board added to a computing device or as a virtual machine. In some embodiments, the control system 140 can be included as part of workstation 102, follower device 104, and/or be operated separately from, and in coordination with, workstation 102 and/or follower device 104. In some embodiments, memory 160 includes one or more non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, any other memory chip, cartridge, module etc.), a communication interface (e.g., Bluetooth interface, infrared interface, network interface, optical interface, etc.), and numerous other elements and functionalities. The non-persistent storage and persistent storage are examples of non-transitory, tangible machine readable media that can include executable code that, when run by one or more processors (e.g., processor 150), can cause the one or more processors to perform one or more of the techniques disclosed herein, including the processes of method 500 and/or the processes of FIGS. 5-9, described below. In addition, functionality of control module 170 can be implemented in any technically feasible software and/or hardware in some embodiments.

Processor 150 can include one or more integrated circuit for processing instructions. For example, the one or more integrated circuits can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, keyboard, mouse, microphone, touchpad, electronic pen, or any other type of input device.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein.

For example, different types of teleoperated systems having follower devices at worksites, as well as non-teleoperated systems, can make use of features described herein.

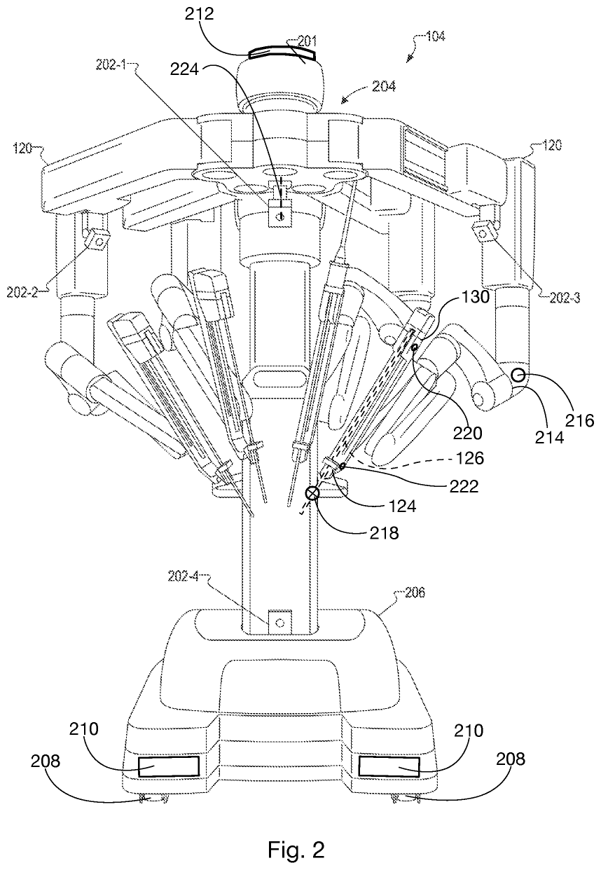

FIG. 2 depicts an example computer-assisted system with an illustrative configuration of a sensor system, according to various embodiments. As shown, imaging devices 202 (imaging devices 202-1 through 202-4) are attached to portions of follower device 104. Although described herein with respect to imaging devices as a reference example, in some embodiments, a sensor system can include any technically feasible sensors, such as monoscopic and stereoscopic optical systems, ultrasonic systems, depth cameras such as cameras using time-of-flight sensors, LIDAR (light detection and ranging) sensors, etc. that are mounted on a computer-assisted system and/or elsewhere. For example, one or more sensors can be mounted on a base, on an orientation platform 204, and/or on one or more manipulator arms 120 of follower device 104. As another example, one or more sensors can be worn by an operator or mounted to a wall, a ceiling, the floor, or other equipment such as tables or carts.

Illustratively, imaging device 202-1 is attached to orientation platform 204 of follower device 104. The orientation platform 204 is part of the repositionable structures of follower device 104 and can be used to simultaneously position and/or orient the manipulator arms 120 relative to a moveable base 206 of follower device 104. Imaging device 202-2 is attached to a first manipulator arm 120 of follower device 104, imaging device 202-3 is attached to a second manipulator arm 120 of follower device 104, and imaging device 202-4 is attached to the moveable base 206. In implementations in which follower device 104 is positioned proximate to a patient (e.g., as a patient side cart), placement of imaging devices 202 at strategic locations on follower device 104 provide advantageous imaging viewpoints proximate to a patient and areas around a worksite where a surgical procedure is to be performed on the patient.

The placements of imaging devices 202 on components of follower device 104 as shown in FIG. 2 are illustrative. Additional and/or alternative placements of any suitable number of imaging devices 202 and/or other sensors on follower device 104, other components of computer-assisted system 100, and/or other components (not shown) located in proximity to the follower device 104 can be used in sensor systems in other embodiments.

Imaging devices 202 and/or other sensors can be attached to components of follower device 104, other components of computer-assisted system 100, and/or other components in proximity to follower device 104 in any suitable way. Additional computer-assisted systems including sensor systems that include sensors are described in International Application Publication No. WO 2021/097332, filed Nov. 13, 2020, and titled “Visibility Metrics in Multi-View Medical Activity Recognition Systems and Methods,” which is hereby incorporated by reference herein.

The sensor system can include various sensors for detecting collisions of any portion of the follower device 104 with one or more obstacles located in proximity to follower device 104. As an example, the moveable base 206 can have one or more proximity sensors 210 mounted thereto. The proximity sensors 210 can be contact or non-contact sensors, and utilize capacitive, inductive, resistive, ultrasonic, or other technology. The moveable base 206 can be mounted to wheels 208 that are actuated according to the methods described herein below and therefore there is a potential for collisions. In other embodiments, the moveable base 206 is repositioned by means of actuated legs, tracks, or other ground-engaging actuation device. In some embodiments, the moveable base 206 moves along rails mounted to a surgical table, wall ceiling, or floor. The moveable base 206 can further include brakes, clamps, or other devices that engage the wheels 208 or other actuators when the moveable base 206 is not being deliberately moved to prevent inadvertent movement of the moveable base 206. In some embodiments, retractable feet can be lowered and placed in contact with a surface supporting the moveable base 206 when the moveable base 206 is not being deliberately moved to prevent inadvertent movement. The sensors for collision may include various sensors for detecting collisions indirectly. For example, joint encoders, force or torque sensors on actuators that drive joints, strain gauges on links, cameras or other imaging devices able to capture images of the system, and/or the like can be used to detect that a joint or actuator is not able to move as commanded, or encounters greater resistance than predicted, or the like. The system may be configured to interpret such responses, or patterns of such response (e.g. particular force profiles, torque values, etc.) as to indicate a collision or to determine characteristics of the collision such as location or speed.

One or more proximity sensors 212 can also be mounted to the follower device 104 at elevated positions, e.g., on or above the orientation platform 204, to detect actual or potential collisions with overhead objects, e.g., light fixtures, that can result from automated movement of the moveable base 206. The proximity sensors 210, 212 can provide sensor data to the control system 140 that are used by the control system 140 to detect and react to collisions or potential collisions. Proximity sensors 210, 212 may additionally or alternatively be mounted to any other joint or link of the follower device 104.

FIG. 2 further illustrates manual controls that can be present on the follower device 104. For example, each manipulator arm 120 can have one or more joints 214. Any of the one or more joints 214 can have an input 216 positioned adjacent thereto, such as positioned on a link joined by the joint 214 to another link. Upon detecting interaction with the input 216, the joint 214 can released the control system 140. The control system 140 can respond to input from input 216 by releasing the joint 214. Releasing the joint 214 can include disengaging a locking mechanism and/or commanding an actuator to disengage or otherwise permit free movement of the joint in response to manual actuation by an operator.

In the illustrated embodiment, the input 216 is positioned adjacent a joint 214 that defines an axis of rotation that substantially intersects, e.g., within 1 mm, a remote center of motion 218 of the manipulator arm 120 including the joint 214 and input 216. The remote center of motion 218 can be defined as a point relative to the cannula mount 124 relative to which translation of the instrument 126, when present, is substantially prevented (e.g., less than 10 mm) while still permitting rotation, e.g., pivoting, of the instrument 126 about the remote center of motion 218. In a medical embodiment where the remote center of motion 218 is placed in an entry to patient anatomy, the manipulator motion at and near the remote center of motion 218 is limited. This positioning can help limit stress on the anatomy of a patient due to manipulator motion at or near the remote center of motion 218. The remote center of motion 218 can be (a) mechanically enforced by configuring the axes of rotation of joints and links of the manipulator arm 120 such that motion of those joints can only cause rotation about the remote center of motion 218, or (b) enforced by the control system 140 commanding only joint movements that will not cause translational movement about the remote center of motion 218, or (c) a combination of some joints that can only cause rotation about the remote center of motion 218, with commands to other joints so as not to move the remote center. For example, mechanical enforcement can be accomplished by the joint 214, and possibly one or more other joints of the manipulator arm 120, having axes of rotation that substantially intersect the remote center of motion 218. In some examples, in response to receiving an indication to move the instrument 126 supported by the manipulator arm 120, the control system 140 commands movements that pivot the instrument 126 about the remote center of motion 218.

The manipulator arm 120 can further include a port clutch input 220 and a cannula latch input 222. When pressed by an operator, the port clutch input 220 instructs the control system 140 to permit translation of the remote center of motion 218. For example, the port clutch input 220 can be pressed in order to permit movement of the manipulator arm 120 into position in order to engage the cannula mount with a cannula inserted into a patient. When pressed by an operator, the cannula latch input 222 mechanically or electronically (e.g., by way of the control system 140) disengages the cannula mount 124 from the cannula. In the illustrated embodiment, the port clutch input 220 and cannula latch input 222 are mounted to a portion of the manipulator arm 120 that holds and actuates the instrument 126. For example, the port clutch input 220 can be located on or near (e.g., within 3 cm of) the transmission mechanism 130 whereas the cannula latch input 222 is located on or near (e.g., within 3 cm of) the cannula mount 124.

Any of the inputs 216, 220, 222 can be embodied as a button, switch, pressure-sensitive electronic device, touch-sensitive electronic device, proximity-sensitive electronic device, or other type of input. As discussed in greater detail below, any of the inputs 216, 220, 222, a combination thereof, or some other input device can receive an operator interaction in order to invoke a positioning mode or a positioning and orienting mode. Other possible input devices can include a camera and a corresponding processor configured to detect command gestures of an operator, a microphone and corresponding processor configured to detect voice commands, a touch screen, or the like.

In the positioning mode or the positioning and orienting mode, an operator can direct the control system 140 to change the position of the moveable base 206 and/or the repositionable structures of the follower device 104 to facilitate positioning and/or orienting of the follower device 104 relative to a patient and a cannula fitted to the patient. For example, the positioning mode or the positioning and orienting mode as described below can be used to guide the moveable base 206, and possibly one or more other joints of the follower device to position the orientation platform 204 and/or orient a front face vector 224 of the orientation platform 204 in a desired relationship relative to a control feature associated with a distal portion of the repositionable structures of the follower device 104, such as a remote center of motion of a manipulator arm 120, a portion of a cannula that is to be later mounted to the cannula mount 124, an instrument 126 to be later mounted to the manipulator arm 120, and/or the like as described in greater detail below.

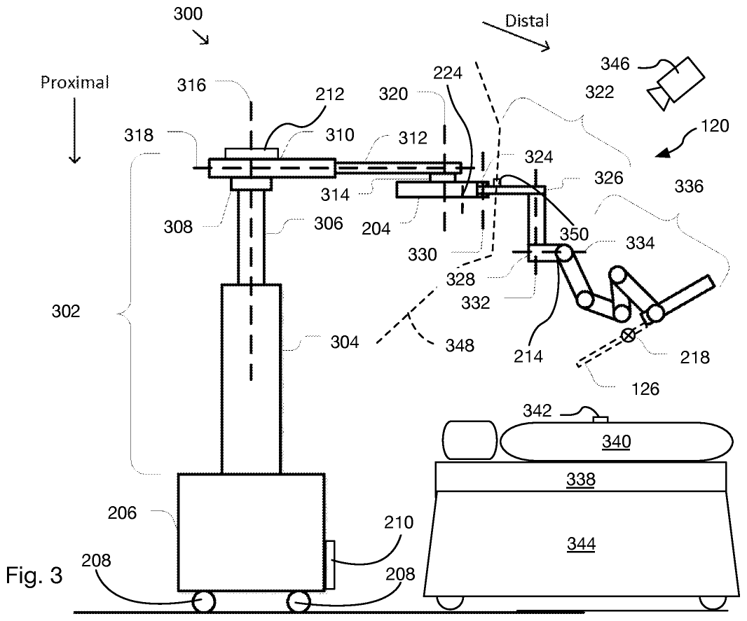

FIG. 3 is a simplified diagram of the computer-assisted system 300 according to some embodiments. As shown in FIG. 3, the computer-assisted system 300 includes a manipulating device comprising multiple repositionable structures. Each repositionable structure shown includes various links and joints. In the following description, the repositionable structures of the computer-assisted system 300 are described in terms of three repositionable structures: a set-up structure 302, a series of set-up joints 322 coupling corresponding links, and a manipulator 336. Although these repositionable structures are described as separate structures, in some instances the set-up structure 302 and the set-up joints 322 and the associated links form a single repositionable structure, or the set-up joints 322 and the associated links and the manipulator 336 form a single repositionable structure. The most proximal portion of the computer-assisted system 300 is the moveable base 206. The set-up structure 302 is mounted to the moveable base 206. Coupled to a distal end of the set-up structure 302 is the series of set-up joints 322. And coupled to a distal end of the set-up joints 322 is the manipulator 336. In some examples, the series of set-up joints 322 and the manipulator 336 correspond to one of the manipulator arms 120. Computer-assisted system 300 is shown with only one set-up structure 302, one series of set-up joints 322, and one corresponding manipulator 336. However, one of ordinary skill would understand that the computer-assisted system 300 can include none or any number of set-up structures, series of set-up joints and associated links, and/or manipulator. Further, example systems can include one or more additional repositionable structures proximal to set-up structure 302, distal to manipulator 336, and/or intervening between the set-up structure 302 and the manipulator 336. In the example shown, the computer-assisted system 300 is equipped with manipulator arms 120 in a fashion that is consistent with the follower device 104. For example, the orientation platform 204 can be secured to the distal end of the set-up structure 302 with the multiple manipulator arms 120 coupled to the orientation platform 204 as shown in FIGS. 1 and 2.

As noted above, the computer-assisted system 300 includes a moveable base 206 that can be repositioned. The moveable base 206 enables the computer-assisted system 300 to be transported from location to location, such as between operating rooms or within an operating room to better position the computer-assisted system 300 near a table 338 bearing a patient 340. The table 338 can include a curtain or drape 344 extending downwardly therefrom. Other drapes can also be present to shield a patient 340 (or inanimate workpiece) from contamination. For example, a drape 348 can be used to isolate portions of the computer-assisted system 300 from patient 340. In the illustrated embodiment, the manipulator arms 120 protrude through the drape 348 and the one or more drape sensors 350 on one or more of the manipulator arms 120 are used to determine whether a drape 348 is present and mounted to cover the moveable base 206, set-up structure 302, boom link 312, orienting platform 204, and/or other portions of the follower device 104, set-up joints 322, or manipulator 336. For example, the drape 348 can have one or more integrated magnets to secure the drape 348 to the manipulator arms 120 and the drape sensors 350 can sense whether the magnets are present. In some embodiments, the drape 348 is mounted to one or more locations on one or more portions of the follower device 104, a separate drape support, the walls, ceiling, or floor of an operating room, the table 338, and/or some other structure.

In some implementations, one or more imaging devices 346 have some or all of the follower device 104 in a field of view thereof. The outputs of the one or more imaging devices 346 can be used to detect possible collisions when moving the moveable base 206 and/or other portions of the computer-assisted system 300. The data from the one or more imaging devices 346 can be additionally or alternatively be used to determine the pose of one or more joints and links of any of the repositionable structures of the computer-assisted system 300.

The set-up structure 302 is mounted on the moveable base 206. As shown in FIG. 3, the set-up structure 302 includes a two-part column including column links 304 and 306. Coupled to the upper or distal end of the column link 306 is a shoulder joint 308. Coupled to the shoulder joint 308 is a two-part boom including boom links 310 and 312. At the distal end of the boom link 312 is a wrist joint 314, and coupled to the wrist joint 314 is the orientation platform 204.

The links and joints of the set-up structure 302 include various degrees of freedom for changing the position and orientation (i.e., the pose) of the orientation platform 204. The two-part column is used to adjust a height of the orientation platform 204 by moving the shoulder joint 308 up and down along an axis 316. The orientation platform 204 is additionally rotated about the moveable base 206, the two-part column, and the axis 316 using the shoulder joint 308. The horizontal position of the orientation platform 204 is adjusted along an axis 318 using the two-part boom. And the orientation of the orientation platform 204 is adjusted by rotation about an axis 320 using the wrist joint 314. Thus, subject to the motion limits of the links and joints in the set-up structure 302, the position of the orientation platform 204 can be adjusted vertically above the moveable base 206 using the two-part column. The position of the orientation platform 204 can also be adjusted radially and angularly about the moveable base 206 using the two-part boom and the shoulder joint 308, respectively. And the angular orientation of the orientation platform 204 can also be changed using the wrist joint 314.

The orientation platform 204 is used as a mounting point for the one or more manipulator arms 120. The ability to adjust the height, horizontal position, and orientation of the orientation platform 204 about the moveable base 206 provides a flexible repositionable set-up structure for positioning and orienting the one or more manipulator arms 120 about a work space, such as a patient, located near the moveable base 206. FIG. 3 shows a single articulated manipulator arm 120 coupled to the orientation platform using a first set-up joint 324. And although only one manipulator arm 120 is shown, one of ordinary skill would understand that multiple manipulator arms 120 can be coupled to the orientation platform 204 using additional first set-up joints.

The first set-up joint 324 forms the most proximal portion of the set-up joints 322 section of the manipulator arm 120. The set-up joints 322 further include a series of joints and links. As shown in FIG. 3, the set-up joints 322 include at least links 326 and 328 coupled via one or more joints (not expressly shown). The joints and links of the set-up joints 322 include the ability to rotate the set-up joints 322 relative to the orientation platform 204 about an axis 330 using the first set-up joint 324, adjust a height of the link 328 relative to the orientation platform along an axis 332, and rotate the manipulator 336 at least about an axis 334 at the distal end of the link 328. The set-up joints 322 can further include additional joints, links, and axes permitting additional degrees of freedom for altering a pose of the manipulator 336 relative to the orientation platform 204.

The manipulator 336 is coupled to the distal end of the set-up joints 322, such as by the joint 214, and includes additional links and joints that permit control over a pose of an end effector or instrument 126 mounted at a distal end of the manipulator 336. In some embodiments, the degrees of freedom in the manipulator 336 permit at least control of the roll, pitch, and yaw of the instrument 126 relative to the distal end of the set-up joints 322. In some examples, the degrees of freedom in the manipulator 336 can further include the ability to advance and/or retreat the instrument 126 about a longitudinal axis of the instrument 126. In some examples, the degrees of freedom of the set-up joints 322 and the manipulator 336 are further controlled so as to maintain the remote center of motion 218 at a point on the instrument 126 or a point on a cannula (not shown). In some examples, the remote center of motion 218 corresponds to a surgical port in a patient so that as the instrument 126 is used, the remote center of motion 218 remains stationary to limit stresses on the anatomy of the patient at the remote center of motion 218. In some examples, the manipulator 336 is consistent with a universal surgical manipulator for use with the da Vinci® Surgical System commercialized by Intuitive Surgical, Inc. of Sunnyvale, California. In some examples, the instrument 126 can be an imaging device such as an endoscope, a gripper, a stapler, an energy delivery instrument, a cutting instrument, any other type of surgical tool, and/or a combination of thereof.

Given the large number of degrees of freedom in the set-up structure 302, the set-up joints 322, and the manipulator 336, it is not always an easy task to determine the best positions of each of the joints so that a pose of the manipulator 336 and, more importantly, a position and/or orientation of the instrument 126 are maintained as desired. Further, as the desired positions and/or orientations of the manipulator 336 and the instrument 126 are adjusted during operation of the computer-assisted system 300, suitable flexibility in ranges of motion and orientation should be available so that the desired positions and/or orientations can be obtained without reaching a range of motion limit on any of the joints in the set-up joints 322 or the manipulator 336. To accomplish this, in some embodiments, positions and/or orientations of the moveable base 206, the set-up structure 302, and/or the orientation platform 204 are typically controlled during a set-up phase or targeting operation so that the joints in the set-up joints 322 and the manipulator 336 are located near a center of their respective ranges of motion while at the same time establishing or maintaining a desired geometric relationship between a reference feature of the set-up structure 302 or the orientation platform 204 relative to a control feature associated with a distal portion of the repositionable structures of the follower device, such as a remote center of motion 218 of a manipulator arm 120, a portion of a cannula that is to be later mounted to a cannula mount of the manipulator arm 120, an instrument 126 to be later mounted to the manipulator arm 120, and/or the like. In some embodiments, this geometric relationship corresponds to positioning the reference feature (e.g., an approximate rotational center point, the axis 320 about which the wrist joint 314 rotates) vertically over the control feature. In some embodiments, the geometric relationship further corresponds to rotating a portion of the set-up structure 302 or the orientation platform 204 to orient the reference feature (e.g., as defined by a front face vector of the orientation platform 204) to achieve a predefined alignment with an axis associated with the control feature (e.g., an insertion axis associated with the manipulator 336). In some embodiments, the geometric relationship additionally includes a distance of the control feature from the reference feature, such as a height of the control feature above the reference feature.

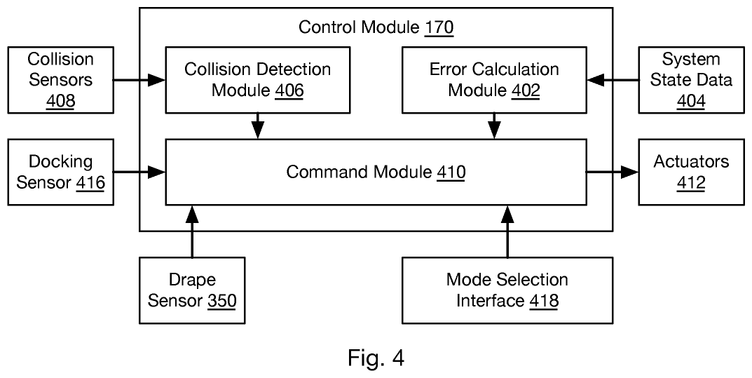

As part of the above-described targeting operation, the moveable base 206 can also be moved toward the patient 340 such that the targeting operation can be performed without exceeding the ranges of motion of some or all of the set-up structure 302 and/or set-up joints 322. FIGS. 4 through 9 illustrate example implementations of positioning modes that can be used to implement the targeting operation and/or the guiding of the moveable base 206 into a desired position relative to the patient 340.

As described above, the positioning modes are performed with respect to a control feature and a reference feature of the repositionable structures of the computer-assisted system 300. In some embodiments, the control feature is manually repositioned by an operator in a world frame that is fixed relative to the environment of the computer-assisted system 300, such as a frame of reference associated with a room (e.g., an operating room) the computer-assisted system 300 is located in. The operator provides an indication (e.g., using one or more of the inputs 216, 220, 222) that the computer-assisted system 300 should be placed in one of the positioning modes. When a second indication is not detected, a first positioning mode is entered where the moveable base 206 is moved relative to the world frame and/or the reference feature is moved relative to a base frame that is associated with the moveable base 206 in order to establish and maintain the desired geometric relationship between the control feature and the reference feature. When the second indication is detected, the computer-assisted system 300 is placed in a second positioning mode where the moveable base 206 is kept stationary in the world frame and the reference feature is moved relative to the base frame that is associated with the moveable base 206 to establish and maintain the desired geometric relationship with between the control feature and the reference feature. An example second indication corresponds to a cannula being docked to the distal repositionable structure, such as by being mounted to the cannula mount 124, by being physically coupled to another part of the distal repositionable structure, or the like.

Both the positioning modes operate in two phases. In a first phase the reference feature is initially moved to the desired geometric relationship relative to the control feature. Once the desired geometric relationship is achieved, a second phase begins where additional repositioning of the control feature by the operator causes an initial displacement of the control feature relative to the reference feature (e.g., as defined by the desired geometric relationship) to change to a new displacement so that the geometric relationship between the control feature and the reference feature is no longer the desired geometric relationship. The control feature is then moved relative to the base frame in order to reduce the difference between the new displacement and the initial displacement and to keep the desired geometric relationship between the control feature and the reference feature. Both the first and second phases operate in a similar fashion by determining how the current geometric relationship between the control feature and the reference feature differ from the desired geometric relationship and moving the reference feature so that the geometric relationship between the control feature and the reference feature is consistent with the desired geometric relationship.

The positioning modes can be understood with different portions of the computer-assisted system 300 performing different actions. moveable base. These different portions include a proximal repositionable structure and a distal repositionable structure. The proximal repositionable structure is supported by the moveable base 206. The distal repositionable structure comprises proximal and distal portions. A proximal portion of the distal repositionable structure is physically supported by the proximal repositionable structure; the proximal portion may be directly coupled to the proximal repositionable structure, or indirectly coupled to the proximal repositionable structure, through one or more intervening components.

A control feature is defined relative to the distal portion of the distal repositionable structure, and a reference feature is defined relative to the proximal repositionable structure. Repositioning of the distal portion of the distal repositionable structure is facilitated by the control system 140 in the positioning modes, such as by allowing one or more of the joints of the distal repositionable structure to be manually moved by the operator. The control system 140 further actuates the moveable base 206 and possibly also the proximal repositionable structure in order to move the reference feature relative to the control feature. As an example, the control feature can be the remote center of motion 218 and the reference feature can be the axis 330 (see FIG. 3).

Using the computer-assisted system 300 as an example, the proximal repositionable structure can include one or more joints and links of the set-up structure 302 and possibly one or more other joints and links positioned between the set-up structure 302 and the manipulator 336. The distal repositionable structure can include the manipulator 336 and one or more additional joints and links between the manipulator 336 and the set-up structure 302. Likewise, the distal portion of the distal repositionable structure can include a distal portion of the manipulator 336.

In some embodiments, the proximal repositionable structure includes the set-up structure 302 and the orientation platform 204 and any intervening joints and links as described above. The reference feature can be selected to be an approximate rotational center point of the orientation platform 204, the axis 320 about which the wrist joint 314 rotates, and/or some other feature of the orientation platform 204. The distal repositionable structure in this example includes the manipulator arm 120. The proximal portion of the distal repositionable structure includes the set-up joints 322 with the remainder of the manipulator arm 120 between the set-up joints 322 and the manipulator 336 and the manipulator 336 itself forming the distal portion of the distal repositionable structure. Alternatively, the entire manipulator arm 120 can be the proximal portion with only the manipulator 336 being the distal portion. The control feature can be selected to be the remote center of motion 218 as defined above.

The joints and links of the computer-assisted system 300 (and/or the follower device 104) can also be divided in other ways between the proximal repositionable structure, distal repositionable structure, and the distal and proximal portions of the distal repositionable structure. Likewise, the locations of the control feature and reference feature can be assigned to different locations relative to a manipulating device (such as the follower device 104).

For example, the control feature can be a point, a line, or other feature associated with the distal portion of the distal repositionable structure (e.g., a position of the point in a three-dimensional space relative to world reference frame; or a manipulating device reference frame, such a reference frame of the moveable base 206). As such, the control feature can be the remote center of motion 218 defined by the distal repositionable structure. The control feature can be point coincident with a location of the instrument 126 or accessory (e.g., cannula 342) when such is mounted to the distal portion (e.g., a point on a longitudinal axis defined relative to the distal portion, which would coincide with a shaft axis of the instrument 126). The control feature can be a point on an axis along which the distal portion can insert or retract the instrument 126 relative to a workspace. The control feature can be a point on a roll axis assoicate with the distal portion, such as expected roll axis of the instrument if mounted, or an expected roll axis of the cannula 342 if mounted. The control feature can be a rotational axis of the distal portion. The control feature can be a portion of a link of the distal portion. The control feature can be determined from one or kinematic models of some or all of the moveable base, the proximal repositionable structure, the distal repositionable structure, the distal portion, the instrument 126, and/or the cannula 342.

In some implementations, the control feature is defined with respect to only one manipulator arm 120 of multiple manipulator arms mounted to the orientation platform 204 such that only one manipulator arm 120 is moved by an operator in the positioning modes. Accordingly, in such implementations, only the inputs 216, 220, and/or 222 of that manipulator arm 120 are used by an operator to provide the indication that the positioning modes should be entered.

The reference feature can be any point, line, or other feature on or near the proximal repositionable structure. For example, the reference feature can be a center point or other point on a link of the proximal repositionable structure (e.g., the orientation platform 204). The reference feature can be a center point or other point on a joint (e.g., the wrist joint 314 linking the boom link 312 to the orientation platform 204). The reference feature can be a point on a rotational axis of a link or joint of the proximal repositionable structure. The reference feature can be a rotational axis of of the proximial repositionable structure. The reference feature can be a portion of a link of the proximal repositionable structure.

The computer-assisted system 300 (and/or follower device 104) itself can have various configurations of joints and links other than those shown in FIG. 13 while still implementing the positioning modes as described herein. Likewise, a manipulating device or follower device can include, or be composed entirely of, an actuatable structure that does not use links and joints, such as flexible pneumatic or hydraulic actuators, compliant mechanisms, and/or other repositionable structures.

FIG. 4 illustrates an example of the control module 170 from FIG. 1 in greater detail, according to various embodiments. The illustrated control module 170 is one example of a hardware module, a software module executed by a control system (e.g., by processor 150 of control system 140), or a combined hardware and software module that can perform some or all of the functions. As shown in FIG. 4, the control module 170 includes, without limitation, an error calculation module 402, a collision detection module 406, and a command module 410. One skilled in the art will understand that the control module 170 can have more or fewer such internal modules, and take in more or fewer inputs. For example, in some instances, the control module 170 does not have a collision detection module. As another example, in some instances, the collision sensors 408 provide data directly to the command module 410. As yet another example, in various instances, the command module does not receive data from docking sensors, drape sensors, etc. Similarly, in various instances, the system may have none, some, or of the sensors shown in FIG. 4, or different sensors than those shown in FIG. 4.

In the example shown in FIG. 4, the error calculation module 402 takes as inputs system state data 404. The system state data 404 can indicate the state of the joints and links of the follower device 104 as indicated by sensors included in the follower device or some other means, such as by analyzing the data from the one or more imaging devices 346. The error calculation module 402 determines, from the system state data 404, a position of the control feature and the reference feature.

The collision detection module 406 receives outputs from one or more collision sensors 408. The collision sensors 408 can include any of the proximity sensors 210, 212 or other collision sensors mounted to the follower device 104, the table 338, or elsewhere. The collision sensors 408 may include joint encoders, torque sensors, or other sensors incorporated into the follower device 104 or able to detect follower device 104, the outputs of which may be processed to determine whether a collision has occurred. The collision sensors 408 can additionally and/or alternatively include the one or more imaging devices 346 and images from the one or more imaging devices 346 can be processed by the collision detection module 406 to detect collisions and potential collisions. For example, the collision detection module 406 can use object detection, object segmentation, classical computer vision techniques for part/object detection, and/or part segmentation techniques to determine the poses of objects and/or portions thereof. In some embodiments, objects and/or portions of objects can be outside the field of view of the sensor(s). In such cases, techniques known in the art, such simultaneous localization and mapping (SLAM), can be employed to determine the objects and/or portions thereof in a reference frame associated with the sensor(s). Additional and/or alternative techniques for detecting objects and/or portions thereof using sensors are described in International Publication No. WO 2022/104118, filed Nov. 12, 2021, and titled “Visibility Metrics in Multi-view Medical Activity Recognition Systems and Methods,” International Publication No. WO 2022/104118, filed Nov. 12, 2021, and titled “Multi-view Medical Activity Recognition Systems and Methods,” and International Publication No. WO 2022/104129, filed Nov. 12, 2021, and titled “Multi-view Medical Activity Recognition Systems and Methods,” which are hereby incorporated by reference herein.

The collision detection module 406 evaluates the outputs of the collision sensors and produces an alert in the event that the outputs indicate that a collision has occurred or is likely to occur. The collision detection module 406 can determine that a collision is likely based on outputs of one or both of the proximity sensors 210, 212 indicating proximity or contact. The collision detection module can determine that a collision is likely or has occurred by evaluating images received from the one or more imaging devices 346 to determine the speed and/or position of structures of the follower device 104 as well as the positions of the patient 340, table 338, and/or other objects or persons in proximity to follower device 104 to determine whether a collision has occurred or is likely to occur.

The collision detection module 406 can additionally detect actual or potential configurations of the follower device 104 that are unacceptable and, in response, provide an output indicating an actual or potential collision. For example, in some scenarios, if the manipulator arms 120 are spread out, moving the set-up structure 302 can cause a manipulator arm 120 to strike the column links 304, 306.

The command module 410 generates commands that are then transmitted to actuators 412. The actuators 412 form an actuator system including some or all of the actuators for joints of the follower device 104 and actuators for the wheels 208 of the moveable base 206. The command module 410 receives outputs from the error calculation module 402 and the collision detection module 406 and the system state data 404. The command module 410 can further utilize one or more kinematic models of the follower device 104 to determine relationships between the system state data 404 and corresponding positions and orientations of various portions of the repositionable structures of the follower device 104. The one or more kinematic models can also be used to determine how to drive the actuators controlling the joints of the follower device 104 to position or orient the various portions of the repositionable structures of the follower device 104.

In some embodiments, the command module 410 transforms the positions and orientations of the various portions of the repositionable structures of the follower device 104 to one or more common reference frames. In some examples, the common reference frames include a base reference frame of the proximal repositionable structure, e.g., the base frame of the moveable base 206, a world frame associated with the environment where the follower device 104 is located, a reference frame associated with a portion of the repositionable structures of the follower device 104, and/or the like. In some embodiments, the command module 410 additionally uses one or more registration techniques to determine geometric relationships between the various sensors and the follower device 104.

In some embodiments, the command module 410 further receives the data from a docking sensor 416 and the one or more drape sensors 350. Sensor data can be in the form of one or more sensor signals; such sensor data can be from some or all of the previously mentioned sensors. The command module 410 can use the output from the docking sensor 416 to determine whether a cannula 342 is mounted to the cannula mount 124 so as to determine whether the first or second positioning modes is to be entered. The docking sensor 416 can be positioned within or mounted to the cannula mount 124 and detect when the cannula mount 124 is engaged with the cannula 342. In some embodiments, the command module 410 limits the speed of movement of the follower device 104 when the drape sensor 350 indicates that a drape 348 is present. This can help reduce accidental collisions during operation, help avoid tearing the drape 348 and provide time for an operator to rearrange the drape 348 where necessary, etc.

The command module 410 is further coupled to a mode selection interface 418. The mode selection interface 418 receives operator interactions providing the indication to enter one of the positioning modes. The mode selection interface 418 can include some or all of the input 216, port clutch input 220, and cannula latch input 222. In some embodiments, the mode selection interface 418 includes a touch screen, a voice processor system and microphone, a gesture detection system and an imaging device, and/or some other device. In some examples, when the mode selection interface 418, is controlled by touch, and the distal portion of the distal repositionable structure and the mode selection inputs are located such that both are simultaneously within reach of the operator.

FIG. 5 is a flow diagram of example method steps for entering a positioning mode for a computer-assisted system, according to various embodiments. Although the method steps are described in conjunction with the embodiments of one or more of FIGS. 1-4, persons of ordinary skill in the art will understand that any system configured to perform the method steps 502-510, in any order, is within the scope of the present disclosure. In some embodiments, the method steps 502-510 can be performed by a module, such as the control module 170 and/or command module 410 in order to determine whether to initiate or continue with a positioning mode.

As shown, a method 500 begins at step 502, the command module 410 determines whether an operator has provided an indication for entering a positioning mode. In some embodiments, step 502 includes evaluating whether an input received through the mode selection interface 418 indicates that the operator would like to enter on of the positioning modes. For example, in one implementation, the operator provides the indication to enter the positioning mode by simultaneously contacting the port clutch input 220 and cannula latch input 222, e.g., simultaneously presses a pair of buttons embodying the inputs 220, 222. In another example, a user invokes the positioning modes by contacting one or more of the inputs 216, 220, and/or 222.

In some embodiments, step 502 further includes evaluating whether an operator has provided an indication that the positioning mode that was previously entered should be exited. An operator can exit the positioning modes in various ways. In some examples, the positioning modes are enabled only while the operator is interacting with the mode selection interface 418 to provide the indication to enter the positioning mode. For example, the manipulating device remains in the positioning mode only while the user is simultaneously activating the one or more inputs used to provide the indication to enter the positioning mode. Accordingly, in such embodiments, the positioning mode is exited as soon as the operator releases or deactivates the inputs used to enter the positioning mode. In some examples, the operator exits the positioning mode using an exit input or exit control provided via the mode selection interface 418. In some examples, the manipulating device exits the positioning mode when movement commands for the moveable base 206 and/or the manipulating device are received from another operator. For example, another operator can provide one or more movement commands using one or more controls located on the moveable base 206 and/or on one of the column links.

When the indication to enter a positioning mode is detected during step 502, the method 500 evaluates one or more criteria before allowing the positioning mode to be entered. For example, at step 504 the command module 410 uses the collision detection module 406 to evaluates whether a collision is predicted or detected. Whether a collision is predicted or detected can be determined by the collision detection module 406 as described above. When a collision is detected or predicted, method 500 repeats the tests of steps 502 and 504 before entering a positioning mode. When no collision is detected or predicted, method 500 proceeds to step 506 to determine which positioning mode is to be entered.

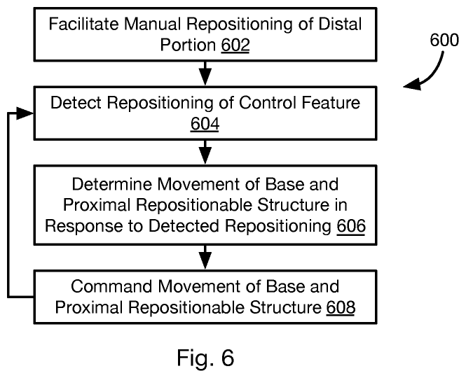

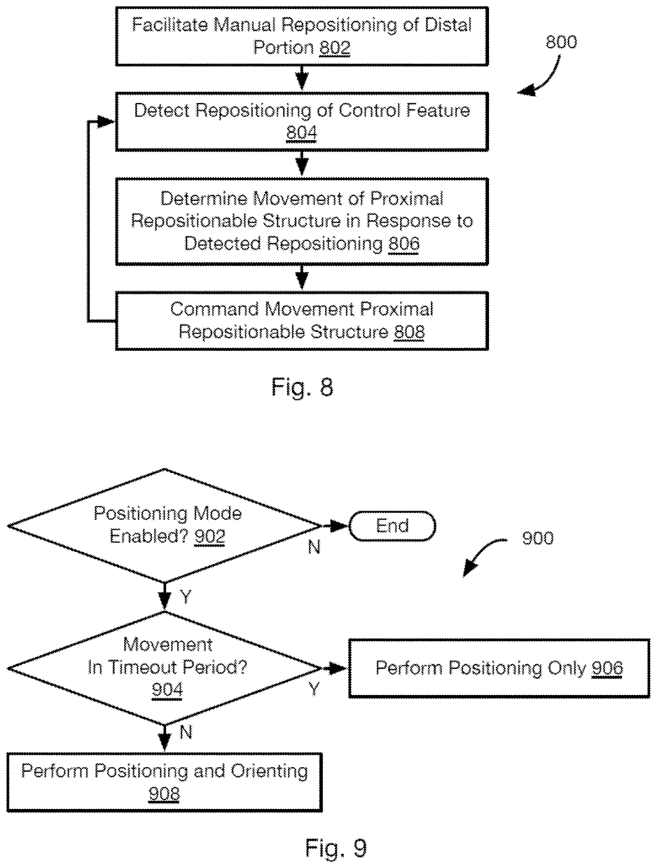

At step 506, the command module 410 determines, whether there is an indication that a cannula is attached directly (or indirectly, such as through a sterile adapter), or otherwise physically coupled, (generally referred to as “docked”) to the manipulating device, such as to cannula mount 124. When the command module 410 does not detect the indication that a cannula is docked, the manipulating device is placed in the first positioning mode at step 510. The first repositioning mode is described in further detail as method 600 in FIG. 6. When the command module detects the indication that a cannula is docked, the manipulating device is placed in the second repositioning mode at step 508, which includes exiting the first mode if the first mode was previously entered. The second repositioning mode is described in further detail as method 800 in FIG. 8.

As discussed above and further emphasized here, FIG. 5 is merely an example which should not unduly limit the scope of the claims. One of ordinary skill in the art would recognize many variations, alternatives, and modifications. In some embodiments, the criteria evaluated in the method 500 to determine whether to enter one of the positioning modes are exemplary only. Any number of criteria can be required to be met. In some examples, the positioning mode(s) (e.g., the previously described first positioning mode and second positioning mode) can be disabled or ended (e.g., exited from the mode) when the moveable base 206 is being actuated or commanded to move by an operator (such as by using a remote control or controls on a non-sterile side of the follower device 104), or is being physically moved by external forces (such as applied by one or more operators). In some examples, the positioning mode(s) is disabled or ended (e.g., exited from) when an instrument is mounted to the distal portion of the distal repositionable structure. In some embodiments, the first positioning mode (and/or second positioning mode if supported by the system) is not exited when a collision or potential collision is detected; instead, the movement of the moveable base 206 and/or the proximal repositionable structure are limited in response to the collision or potential collision, so that movement in the direction of the collision or potential collision is not performed. As additional examples, an indication not to enter the first positioning mode may comprise any one or more of the example criteria that cause the system to disable or exit the positioning mode (e.g. cannula docked to the distal repositionable structure, instrument mounted to the distal repositionable structure, base being actuated or commanded to move, base being physically moved, actual or potential collision, etc.).

In some embodiments, the response to a collision or potential collision does not vary regardless of if a drape is mounted to the computer-assisted system 300, or to a particular part of the computer-assisted system 300 (e.g. to a part of the follower device 104 if the manipulating device comprises such follower device). In some embodiments, the response to a collision or potential collision varies depending upon whether a drape is mounted to the computer-assisted system 300 (or the follower device 104), upon where the drape is mounted to the computer-assisted system 300 (or the follower device 104), upon what part the drape is mounted to cover of the computer-assisted system 300 (or the follower device 104), etc. In some examples, when a drape is detected and a collision or potential collision is detected, the positioning mode is exited in response to a collision being detected with some portion of the computer-assisted system while a drape is mounted to the computer-assisted system. In some examples, the control system is further configured to exit the positioning mode in response to detecting (using one or more sensors) a collision with the distal repositionable structure while a drape is mounted to cover the distal repositionable structure. In some examples, the control system is further configured to determine whether a collision with the computer-assisted system is associated with a drape mounted to cover at least a portion of the computer-assisted system and exit the first mode in response a determine such a collision has occurred. In some such computer-assisted systems, the control system may not exit the first mode in response to determining that a collision has occurred with a portion of the computer-assisted system not covered by the drape. In some instances, one or more indications are provided to an operator, such as to indicate that there may have been a breach in the sterile barrier.

In some examples, when a drape is not detected when a collision (or potential collision) is detected, movement in a direction of the collision (or potential collision) is not performed while movement in other directions can continue to be performed.

In some embodiments, the response to a collision or potential collision depends on a joint or other portion of the repositionable structure that is associated with the collision or the potential collision. In some examples, the positioning mode is exited when the collision or potential collision is associated with a more proximal joint (e.g., a joint or link in the set-up structure 302) of the manipulating device. In some examples, the positioning mode becomes limited and is not exited. For example, movements in directions other than the direction of the collision (or potential collision) are permitted when the collision (or potential collision) is associated with a more distal joint and/or link. As another example, movement in other than the vertical is permitted when the collision (or potential collision) is associated with a joint and/or link that controls vertical movement of the manipulating device, as long as the relative vertical position does not increase in the direction of the collision.

FIG. 6 is a flow diagram of example method steps for implementing a first positioning mode for a computer-assisted system, according to various embodiments. Although the method steps are described in conjunction with the embodiments of FIGS. 1-4, persons of ordinary skill in the art will understand that any system configured to perform the method steps 602-608, in any order, is within the scope of the present disclosure. In some embodiments, the method steps 602-608 can be performed by a module, such as the control module 170 and/or command module 410.

A method 600 begins at step 602 where the command module 410 facilitates manual repositioning of the distal portion of the distal repositionable structure. Facilitating manual repositioning of the distal portion of the distal repositionable structure includes releasing one or more joints of distal repositioning structure such that an operator can freely move the distal repositionable structure, which also moves the control feature subject to limitations of the range of motion of one or more joints of the distal repositioning structure. The releasing of a joint can include releasing a brake associated with the joint and/or driving the joint based on an actual position and/or velocity of the joint. Techniques for facilitating manual repositioning of a repositionable structure are described in further detail in International Publication No. WO 2015/142930, filed Mar. 17, 2015, and titled “System and Method for Breakaway Clutching in an Articulated Arm,” which is hereby incorporated by reference herein. In the example computer-assisted system 300, step 602 can include releasing one or more joints of the set-up joints 322 and/or some or all of the joints of the manipulator arm 120.

At step 604, the command module 410 detects the repositioning of the control feature relative to the base frame. The repositioning of the distal portion occurs in response to force applied to the distal portion and/or some other portion of the distal repositionable structure by the operator. In a typical use case, the operator will move the distal portion to move the distal portion to the actual or expected location of a cannula 342 to be mounted to the cannula mount 124. The operator can further rotate the distal portion to align the distal portion with the axis of an opening and/or lumen defined by the cannula 342. As the operator repositions the distal portion, the command module 410 determines one or more geometric parameters that characterize the control feature. In some examples, the one or more geometric parameters include a position of the control feature, such as a position of the control feature in a three-dimensional space relative to the world frame or the base frame of the moveable base 206 or a position on a plane that is parallel to a surface (e.g., a floor or a top of a patient table) on which the manipulating device is located and/or otherwise mounted.

In some embodiments, the one or more geometric parameters include information about a control orientation of the control feature. In some examples, the control orientation is defined by an axis associated with the distal portion, such as an axis defined by any of the joints of the distal portion, an axis along which the distal portion can insert or retract an instrument relative to the distal portion, a longitudinal axis of the distal portion, a longitudinal axis of the cannula, a longitudinal axis of a shaft of an instrument to be later mounted to the distal portion, or a roll axis assoicated with the distal portion, the instrument, or the cannula.

At step 606, in response to detecting the repositioning during step 604, the command module 410 determines movement of the moveable base 206 and a structure movement the proximal repositionable structure. The combined movement of the moveable base 206 relative to the world frame and the movement of the proximal repositionable structure relative to the base frame moves the reference feature toward the desired geometric relationship between the control feature and the reference feature so that the geometric relationship between the control feature and the reference feature eventually reaches the desired geometric relationship. And although the command module 410 determines the movement of both the moveable base 206 and the proximal repositionable structure, in some cases, there can be no movement of the moveable base 206 or no movement of the proximal repositionable structure. For example, only the movement of the moveable base 206 occurs or only movement of the proximal repositionable structure occurs.

The desired geometric relationship defines a desired relationship between the one or more geometric parameters of the control feature and corresponding one or more geometric parameters of the reference feature. In some examples, the desired geometric relationship includes positioning the reference feature at a specified distance and/or specified direction relative to the position of the control feature. In some examples, the desired geometric relationship includes a predefined alignment relationship between the control feature and the reference feature. In some examples, the predefined alignment relationship includes positioning the reference feature vertically above the control feature. In some examples, the pre-defined alignment relationship includes positioning the reference feature so that a rotational axis associated with a link or joint that corresponds to the reference feature intersects the position of the control feature.

In some embodiments, the desired geometric relationship optionally includes orienting a reference orientation of the reference feature and/or other portion of the proximal repositionable structure based on the control orientation of the control feature. In some examples, the reference orientation of the reference feature corresponds to an axis perpendicular to the axis used to determine the reference feature, an axis corresponding to a direction relative to the reference feature that is centrally located between two or more distal repositionable structures that are mounted to a link of the proximal repositionable structure (e.g., a front face vector of the orientation platform 204), and/or the like. In some examples, the desired geometric relationship is determined so that a projection of the reference orientation in a plane is in a same direction as a projection of the control orientation in the same plane. In some examples, the plane is the plane that is parallel to the surface on which the manipulating device is located and/or otherwise mounted or a plane perpendicular to the axis used to position the reference feature relative to the control feature.

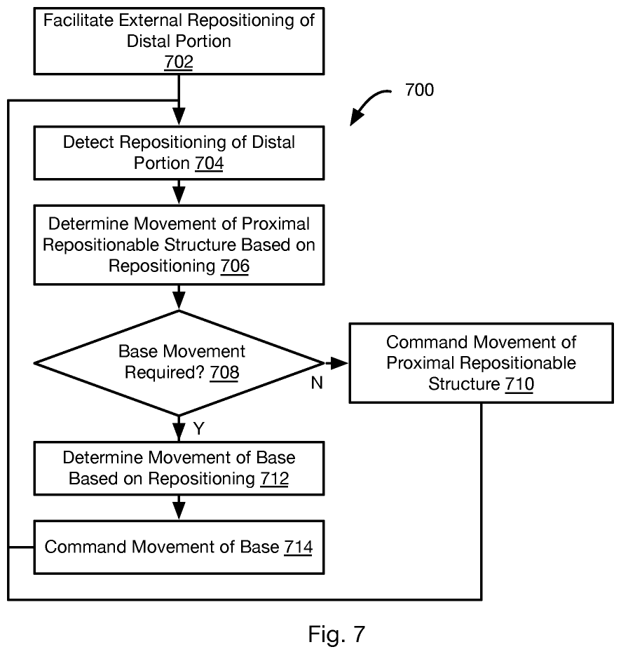

In some embodiments, the partitioning of the movement between the movement of the moveable base 206 relative to the world frame and the proximal repositionable structure relative to the base frame is determined in order to satisfy a predefined criteria set associated with the moveable base or the manipulating device. In some examples, the predefined criteria set includes ensuring that none of the joints in either the proximal repositionable structure or the distal repositionable structure moves within a threshold distance (e.g., within 0, 10, 20, 30, or some other percent of a full range of motion for the joint) from a range of motion limit. In some examples, the predefined criteria set includes centering each joint at a near a center, such as within 10, 20, 30 or some other percent of full range of motion for the joint, of the range of motion for the joint. For example, when one or more of the joints of the proximal repositionable structure reach the threshold distance from their respective range of motion limit, movement of the moveable base can be used to move the reference feature toward the desired geometric relationship between the control feature and the reference feature. In some examples, the predefined criteria set includes ensuring that the moveable base 206 does not collide with or remains a threshold distance from other objects, such as the operating table 338, the patient, the operator, and/or other fixtures located near the manipulating device. In some embodiments, satisfying the predefined criteria set determines whether there is movement of the moveable base 206 relative to the world frame, whether there is movement of the proximal repositionable structure relative to the base frame, or whether there is both movement of the moveable base 206 relative to the world frame and movement of the proximal repositionable structure relative to the base frame. The partitioning of the movement between the movement of the moveable base 206 relative to the world frame and the proximal repositionable structure relative to the base frame is described in further detail in FIG. 7.

At step 608, the command module 410 commands the movement of the moveable base 206 and the proximal repositionable structure according to the corresponding movements determined during step 606. In some embodiments, the command module uses the actuators 412 to cause the movement of the moveable base 206 and the proximal repositionable structure. In some embodiments, one or more criteria are used to determine a speed of the commanded movement of the moveable base 206 and/or the proximal repositionable structure. In some examples, a speed of the movement is lower when the command module 410 determines that a drape is attached to the manipulating device. In some examples, the speed of the commanded movement is based on a difference between the current geometric relationship between the control feature and the reference feature and the desired geometric relationship between the control feature and the reference feature. In some examples, the speed of the commanded movement is based on how close the moveable base 206 and/or other portions of the manipulating device are to objects, such as a patient and/or other personnel, in proximity to the manipulating device.

Method 600 then returns to step 604 to continue to move the reference feature toward the desired geometric relationship between the control feature and the reference feature as further repositioning of the distal portion occurs, such as repositioning of the control feature that causes an initial displacement of the control feature relative to the reference feature to change to a new displacement and causes the geometric relationship between the control feature and the reference feature to no longer be the same as the desired geometric relationship.

FIG. 7 illustrates a more detailed implementation of the method 600 that can be performed in the first positioning mode while accounting for range of motion limits of the manipulating device. Referring specifically to FIG. 7, the command module 410 can execute the method 700 while the first positioning mode is enabled.

The method 700 begins at step 702 where the command module 410 facilitates repositioning of the distal portion. Step 702 can be implemented as described above with respect to step 602. At step 704, the command module 410 detects repositioning of the distal portion. Step 704 can be implemented as described above with respect to step 604.

At step 706, the command module 410 determines movement of one or more joints of the proximal repositionable structure relative to the base frame in response to detecting the repositioning of the control feature relative to the base frame. The movement of the proximal repositionable structure moves the reference feature toward the desired geometric relationship between the control feature and the reference feature so that the geometric relationship between the control feature and the reference feature eventually reaches the desired geometric relationship. As discussed above with respect to step 606, the desired geometric relationship includes one or more of positioning the control feature at a specified distance and/or specified direction relative to the position of the control feature, establishing a predefined alignment relationship between the control feature and the reference feature, and/or orienting a reference orientation of the reference feature and/or other portion of the proximal repositionable structure based on the control orientation of the control feature.

The command module may determine the movement of the one or more joints of the proximal repositionable structure relative to the base frame based on a geometric parameter. The command module may limit, based on a magnitude associated with the first geometric parameter, a movement of the proximal repositionable structure and/or the base. In some embodiments, the geometric parameter may comprise a position, and the magnitude may be a distance between the reference feature and the geometric parameter. In some embodiments, the geometric parameter may comprise a difference, and the command module is configured to determine the magnitude as a magnitude of the difference. For example, the geometric parameter may be a position of the control feature relative to the reference feature.

At step 708, the command module 410 determines whether movement of the moveable base 206 is required. One or more criteria in a predefined criteria set can be evaluated at step 708 to determine whether movement of the moveable base 206 is required. For example, a criteria can include whether one or more joints of the proximal repositionable structure are within a threshold of a range of motion limit. For example, the criteria can include evaluating whether any joint, all joints, or a maximum permitted number of joints, of the proximal repositionable structure has moves within a threshold distance (e.g., within 0, 10, 20, 30, or some other percent of a full range of motion for the joint) from a range of motion limit and/or are at a near a center, such as within 10, 20, 30 or some other percent of full range of motion for the joint, of the range of motion for the joint. The criteria can be evaluated with respect to either the current position and/or orientation of the proximal repositionable structure or the position and/or orientation of the proximal repositionable structure if the movement determined at step 706 is implemented.

If movement of the moveable base 206 is not found to be required at step 708, then the command module 410 commands, at step 710, movement of proximal repositionable structure according to corresponding movments determined during step 706 while maintaining the moveable base 206 stationary. In some embodiments, the command module 410 uses the actuators 412 to cause the movement of the proximal repositionable structure. In some embodiments, one or more criteria are used to determine a speed of the commanded movement of the moveable base 206 and/or the proximal repositionable structure. In some examples, a speed of the movement is lower when the command module 410 determines that a drape is attached to the manipulating device. In some examples, the speed of the commanded movement is based on a difference between the current geometric relationship between the control feature and the reference feature and the desired geometric relationship between the control feature and the reference feature. In some examples, the speed of the commanded movement is based on how close the moveable base 206 and/or other portions of the manipulating device are to objects in proximity to the manipulating device.

If movement of the moveable base 206 is found to be required at step 708, the command module 410 determines, at step 712, a movement of the moveable base 206 relative to the world frame to move the reference feature toward the defined geometric relationship. At step 714, the command module 410 commands movement of the moveable base 206 based on the movement determined during step 712. The movement determined at step 712 and commanded at step 714 can be determined in order to position, orient, or both position and orient the moveable base 206. The movement can include driving the moveable base 206 toward or away from the patient 340, rotating the moveable base 206 to orient an axis of the moveable base (e.g., an axis of the moveable base, an axis of the boom links 310, 312, an axis of the orientation platform 204, or some other portion of the follower device) to move the reference feature to the desired geometric relationship relative to the control feature. The movement can include driving one or more wheels 208 of the moveable base 206 that allows the moveable base 206 to move relative to a floor or other horizontal planar surface. The movement can include driving the moveable base along one or more rails and/or rotating the moveable base 206, or a portion of the moveable base 206, relative to the one or more rails. In some embodiments, one or more criteria are used to determine a speed of the commanded movement of the moveable base 206 and/or the proximal repositionable structure. In some examples, a speed of the movement is lower when the command module 410 determines that a drape is attached to the manipulating device. In some examples, the speed of the commanded movement is based on a difference between the current geometric relationship between the control feature and the reference feature and the desired geometric relationship between the control feature and the reference feature. In some examples, the speed of the commanded movement is based on how close the moveable base 206 and/or other portions of the manipulating device are to objects in proximity to the manipulating device.

Method 700 then returns to step 704 to continue to move the reference feature toward the desired geometric relationship between the control feature and the reference feature as further repositioning of the distal portion occurs, such as repositioning of the control feature that causes an initial displacement of the control feature relative to the reference feature to change to a new displacement and causes the geometric relationship between the control feature and the reference feature to no longer be the same as the desired geometric relationship.

FIG. 8 is a flow diagram of example method steps for implementing the second positioning mode for the manipulating device, according to various embodiments. Although the method steps are described in conjunction with the embodiments of FIGS. 1-4, persons of ordinary skill in the art will understand that any system configured to perform part of all of the method steps 802-808, in any appropriate order, is within the scope of the present disclosure. In some embodiments, the method steps 802-808 can be performed by a module, such as the control module 170 and/or command module 410.

A method 800 begins at step 802, where the command module 410 facilitates manual repositioning of the distal portion of the distal repositionable structure. Step 802 can be implemented as described above with respect to step 602. At step 804, the command module 410 detects repositioning of the distal portion. Step 804 can be implemented as described above with respect to step 604.

At step 806, in response to the detecting the repositioning during step 804, the command module 410 determines movement of the proximal repositionable structure, The movement of the proximal repositionable structure relative to the base frame moves the reference feature toward the desired geometric relationship between the control feature and the reference feature so that the geometric relationship between the control feature and the reference feature eventually reaches the desired geometric relationship. Step 806 can be implemented as described above with respect to step 706.

At step 808, the command module 410 commands the movement of the proximal repositionable structure according to the corresponding movements determined during step 806 while maintaining the moveable base 206 stationary. Step 808 can implemented as described above with respect to step 710.

Method 800 then returns to step 804 to continue to move the reference feature toward the desired geometric relationship between the control feature and the reference feature as further repositioning of the distal portion occurs, such as repositioning of the control feature that causes an initial displacement of the control feature relative to the reference feature to change to a new displacement and causes the geometric relationship between the control feature and the reference feature to no longer be the same as the desired geometric relationship.

FIG. 9 illustrates a flow diagram of example method steps for selecting between positioning or positioning and orienting in the first or second positioning modes, according to various embodiments. Although the method steps are described in conjunction with the embodiments of FIGS. 1-4, persons of ordinary skill in the art will understand that any system configured to perform the method steps 902-908, in any order, is within the scope of the present disclosure. In some embodiments, the method steps 902-908 can be performed by a module, such as the control module 170 and/or command module 410. In some embodiments, the method steps 902-908 are performed to determine whether the movements determined during steps 606, 706, 712, and/or 806 in the first and second repositioning modes should include movement to orient a reference orientation of the reference feature and/or other portion of the proximal repositionable structure based on the control orientation of the control feature as described in further detail above with respect to step 606.

A method 900 begins at step 902 where the command module 410 determines whether either the first positioning mode or the second positioning mode has been entered. Step 902 can be implemented as described above with respect to method 500 of FIG. 5. If the command module 410 determines that neither the first positioning mode nor the second positioning mode have been entered, method 900 repeats step 902 to wait until either the first positioning mode or the second positioning mode has been entered. If the command module 410 determines that either the first positioning mode or the second position mode has been entered, method 900 moves to step 904.

At step 904, the command module 410 determines whether movement of the distal portion occurs during a movement timeout period that begins when the first positioning mode or the second positioning mode is entered. In some examples, the command module 410 determines that movement has not occurred when there is no movement of the distal portion or movement of the distal portions that is less than a threshold displacement and/or a change in orientation of the distal portion that is less than a threshold orientation change. If the command module 410 determines that there is movement of the distal portion during the movement timeout period, method 900 continues at step 906. If the command module 410 determines that there is no movement of the distal portion during the movement timeout period, method 900 continues at step 908.

At step 906, the command module 410 performs only positioning of the reference feature during the first positioning mode or the second positioning mode. For example, the movement determined during steps 606, 706, 712, and/or 806 to move the reference feature toward the desired geometric relationship with the control feature and/or to maintain the desired geometric relationship with the control feature includes only movement to position the reference feature at a specified distance and/or specified direction relative to the position of the control feature and/or to establish or maintain the predefined alignment relationship between the control feature and the reference feature without including movement to orient the reference orientation of the reference feature and/or the other portion of the proximal repositionable structure based on the control orientation of the control feature.

At step 908, the command module performs both positioning and orientating of the reference feature the first positioning mode or the second positioning mode. For example, the movement determined during steps 606, 706, 712, and/or 806 to move the reference feature toward the desired geometric relationship with the control feature and/or to maintain the desired geometric relationship with the control feature includes both movement to position the reference feature at a specified distance and/or specified direction relative to the position of the control feature and/or to establish or maintain the predefined alignment relationship between the control feature and the reference feature and movement to orient the reference orientation of the reference feature and/or the other portion of the proximal repositionable structure based on the control orientation of the control feature.

As discussed above and further emphasized here, FIGS. 5-9 are merely examples which should not unduly limit the scope of the claims. One of ordinary skill in the art would recognize many variations, alternatives, and modifications. In some embodiments, two control features can be defined on two manipulator arms 120 and a combination of the two control features are used to determine a composite control feature. The composite control feature is then used to determine the movement of the reference feature to establish or maintain the desired geometric relationship between the reference feature and the composite control feature. For example, manual movement of the two manipulator arms 120 (e.g., pushing on one manipulator arm while pulling on another manipulator arm) can be used to invoke a rotation of the reference feature (e.g., a rotation of orientation platform 204 and/or movement of the moveable base 206 along an arc.

Although illustrative embodiments have been shown and described, a wide range of modification, change and substitution is contemplated in the foregoing disclosure and in some instances, some features of the embodiments can be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure, such as the various methods, processes, and/or steps, may take the form of an entirely hardware embodiment, an entirely software embodiment (including firmware, resident software, micro-code, etc.) or an embodiment combining software and hardware aspects that may all generally be referred to herein as a “module” or a “computer.” In addition, any hardware and/or software technique, process, function, component, engine, module, or system described in the present disclosure may be implemented as a circuit or set of circuits. Furthermore, appropriate aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus, and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

1. A computer-assisted system comprising:
a base having a base frame;
a proximal repositionable structure supported by the base;
a distal repositionable structure having a proximal portion and a distal portion, wherein the proximal portion is supported by the proximal repositionable structure;
an actuator system configured to move the base, the proximal repositionable structure, and the distal repositionable structure; and
a control system communicatively coupled to the actuator system;
wherein the control system is configured to:
in response to receiving a first indication to enter a first mode while not receiving a second indication not to enter the first mode, enter the first mode; and
wherein the control system is further configured to, while in the first mode:
facilitate external repositioning of the distal portion that moves a control feature relative to the base frame, wherein the control feature is fixed relative to the distal portion,
detect a first repositioning of the distal portion that moves the control feature relative to the base frame,
determine, in response to detecting the first repositioning and based on a first geometric parameter characterizing the control feature after the first repositioning, a first base movement of the base relative to a world frame and a first structure movement of the proximal repositionable structure relative to the base frame, wherein performing the first base movement and the first structure movement together moves a reference feature relative to the control feature, and wherein the reference feature is fixed relative to a portion of the proximal repositionable structure, and
command the actuator system to move the computer-assisted system based on the first base movement and the first structure movement.

2. The computer-assisted system of claim 1, wherein the control system is further configured to:
determine the first geometric parameter as a position of the control feature after the first repositioning.

3. The computer-assisted system of claim 2, wherein to determine the first base movement and the first structure movement, the control system is configured to:
determine the first base movement and the first structure movement to move the control feature to achieve a predefined alignment relationship between the reference feature and the position of the control feature.

4. The computer-assisted system of claim 3, wherein to determine the first base movement and the first structure movement to achieve the predefined alignment relationship, the control system is configured to:
determine that achieving the predefined alignment relationship after the first repositioning while satisfying a predefined criteria set requires moving the proximal repositionable structure relative to the base frame and moving the base relative to the world frame.

5. The computer-assisted system of claim 3, wherein the control system is further configured to, while in the first mode:
detect a second repositioning of the distal portion that moves the control feature relative to the base frame, wherein the predefined alignment relationship can be achieved after the second repositioning while satisfying a predefined criteria set by moving the proximal repositionable structure relative to the base frame while holding the base stationary relative to the world frame;
determine, in response to detecting the second repositioning and based on a second geometric parameter characterizing the control feature after the second repositioning, a second structure movement of the proximal repositionable structure relative to the base frame, wherein performing only the second structure movement moves the reference feature relative to the control feature and achieves the predefined alignment relationship while satisfying the predefined criteria set; and
command the actuator system to move the proximal repositionable structure based on the second structure movement while holding the base stationary in the world frame.

6. The computer-assisted system of claim 3, wherein the control system is further configured to, while in the first mode:
detect a second repositioning of the distal portion that moves the control feature relative to the base frame, wherein, the predefined alignment relationship can be achieved after the second repositioning while satisfying a predefined criteria set by moving the base relative to the world frame while holding the proximal repositionable structure stationary relative to the base frame;
determine, in response to detecting the second repositioning and based on a second geometric parameter characterizing the control feature after the second repositioning, a second base movement of the base relative to the world frame, wherein performing only the second base movement moves the reference feature relative to the control feature and achieves the predefined alignment relationship while satisfying the predefined criteria set; and
command the actuator system to move the base based on the second base movement while holding the proximal repositionable structure stationary in the base frame.

7. The computer-assisted system of claim 1, wherein the control feature has an initial displacement relative to the reference feature at an entry to the first mode, and wherein the control system is further configured to:
determine the initial displacement;
determine, after the first repositioning, a first displacement of the control feature relative to the reference feature; and
determine the first geometric parameter as a difference between the first displacement and the initial displacement.

8-9. (canceled)

10. The computer-assisted system of claim 7, wherein the control system is further configured to, while in the first mode:
detect a second repositioning of the distal portion that moves the control feature relative to the base frame, wherein reducing the difference can be achieved after the second repositioning while satisfying a predefined criteria set by moving the proximal repositionable structure relative to the base frame while holding the base stationary relative to the world frame;
determine, in response to detecting the second repositioning and based on a second geometric parameter characterizing the control feature after the second repositioning, a second structure movement of the proximal repositionable structure relative to the base frame, wherein performing only the second structure movement moves the reference feature relative to the control feature and reduces the difference while satisfying the predefined criteria set; and
command the actuator system to move the proximal repositionable structure based on the second structure movement while holding the base stationary in the world frame.

11. The computer-assisted system of claim 7, wherein the control system is further configured to, while in the first mode:
detect a second repositioning of the distal portion that moves the control feature relative to the base frame, wherein reducing the difference can be achieved after the second repositioning while satisfying a predefined criteria set by moving the base relative to the world frame while holding the proximal repositionable structure stationary relative to the base frame;
determine, in response to detecting the second repositioning and based on a second geometric parameter characterizing the control feature after the second repositioning, a second base movement of the base relative to the world frame, wherein performing only the second base movement moves the reference feature relative to the control feature and reduces the difference while satisfying the predefined criteria set; and
command the actuator system to move the base based on the second base movement while holding the proximal repositionable structure stationary in the base frame.

12-14. (canceled)

15. The computer-assisted system of claim 1, wherein the control system is further configured to limit, based on a magnitude associated with the first geometric parameter, one or more movements selected from the group consisting of: the first base movement or the first structure movement.

16. (canceled)

17. The computer-assisted system of claim 1, wherein the control system is further configured to limit a speed of one or more of the first base movement or the first structure movement based on at least one parameter selected from the group consisting of:
whether a drape is mounted to cover the proximal repositionable structure; or
whether a drape is mounted to cover the distal repositionable structure.

18. The computer-assisted system of claim 1, further comprising:
one or more collision sensors;
wherein the control system is further configured to limit the first base movement or the first structure movement in a first direction associated with a collision detected by the one or more collision sensors, while allowing the first base movement or the first structure movement in a second direction different from the first direction.

19-28. (canceled)

29. A method comprising:
receiving, by a control system, a first indication to enter a first mode with respect to a computer-assisted system, the computer-assisted system comprising:
a base having a base frame;
a proximal repositionable structure supported by the base;
a distal repositionable structure having a proximal portion and a distal portion,
wherein the proximal portion is supported by the proximal repositionable structure;
an actuator system configured to move the base, the proximal repositionable structure, and the distal repositionable structure, the control system being communicatively coupled to the actuator system; and
in response to receiving the first indication and failing to receive a second indication not to enter the first mode, entering, by the control system, the first mode and, while in the first mode:
facilitating, by the control system, external repositioning of the distal portion that moves a control feature relative to the base frame, wherein the control feature is fixed relative to the distal portion,
detecting, by the control system, a first repositioning of the distal portion that moves the control feature relative to the base frame,
determining, by the control system and in response to detecting the first repositioning and based on a first geometric parameter characterizing the control feature after the first repositioning, a first base movement of the base relative to a world frame and a first structure movement of the proximal repositionable structure relative to the base frame, wherein performing the first base movement and the first structure movement together moves a reference feature relative to the control feature, and wherein the reference feature is fixed relative to a portion of the proximal repositionable structure, and
commanding, by the control system, the actuator system to move the computer-assisted system based on the first base movement and the first structure movement.

30. The method of claim 29, further comprising determining, by the control system, the first geometric parameter as a position of the control feature after the first repositioning.

31. The method of claim 30, wherein determining the first base movement and the first structure movement comprises:
determining the first base movement and the first structure movement to move the control feature to achieve a predefined alignment relationship between the reference feature and the position of the control feature.

32. The method claim 31, wherein determining the first base movement and the first structure movement to achieve the predefined alignment relationship comprises:
determining that achieving the predefined alignment relationship after the first repositioning while satisfying a predefined criteria set requires moving the proximal repositionable structure relative to the base frame and moving the base relative to the world frame.

33. The method of claim 31, further comprising, while in the first mode:
detecting, by the control system, a second repositioning of the distal portion that moves the control feature relative to the base frame, wherein the predefined alignment relationship can be achieved after the second repositioning while satisfying a predefined criteria set by moving the proximal repositionable structure relative to the base frame while holding the base stationary relative to the world frame;
determining, by the control system and in response to detecting the second repositioning and based on a second geometric parameter characterizing the control feature after the second repositioning, a second structure movement of the proximal repositionable structure relative to the base frame, wherein performing only the second structure movement moves the reference feature relative to the control feature and achieves the predefined alignment relationship while satisfying the predefined criteria set; and
commanding, by the control system, the actuator system to move the proximal repositionable structure based on the second structure movement while holding the base stationary in the world frame.

34. The method of claim 31, further comprising, while in the first mode:
detecting, by the control system, a second repositioning of the distal portion that moves the control feature relative to the base frame, wherein, the predefined alignment relationship can be achieved after the second repositioning while satisfying a predefined criteria set by moving the base relative to the world frame while holding the proximal repositionable structure stationary relative to the base frame;
determining, by the control system and in response to detecting the second repositioning and based on a second geometric parameter characterizing the control feature after the second repositioning, a second base movement of the base relative to the world frame, wherein performing only the second base movement moves the reference feature relative to the control feature and achieves the predefined alignment relationship while satisfying the predefined criteria set; and
commanding, by the control system, the actuator system to move the base based on the second base movement while holding the proximal repositionable structure stationary in the base frame.

35. The method of claim 29, wherein:
the control feature has an initial displacement relative to the reference feature at an entry to the first mode; and
the method further comprises:
determining, by the control system, the initial displacement;
determining, by the control system and after the first repositioning, a first displacement of the control feature relative to the reference feature; and
determining, by the control system, the first geometric parameter as a difference between the first displacement and the initial displacement.

36-49. (canceled)

50. A non-transitory machine-readable medium comprising a plurality of machine-readable instructions which when executed by one or more processors associated with a computer-assisted device, are adapted to cause the computer-assisted device to perform a method comprising:
receiving a first indication to enter a first mode with respect to a computer-assisted system, the computer-assisted system comprising:
a base having a base frame;
a proximal repositionable structure supported by the base;
a distal repositionable structure having a proximal portion and a distal portion, wherein the proximal portion is supported by the proximal repositionable structure;
an actuator system configured to move the base, the proximal repositionable structure, and the distal repositionable structure, the control system being communicatively coupled to the actuator system; and
in response to receiving the first indication and failing to receive a second indication not to enter the first mode, entering the first mode and, while in the first mode:
facilitating external repositioning of the distal portion that moves a control feature relative to the base frame, wherein the control feature is fixed relative to the distal portion,
detecting a first repositioning of the distal portion that moves the control feature relative to the base frame,
determining, in response to detecting the first repositioning and based on a first geometric parameter characterizing the control feature after the first repositioning, a first base movement of the base relative to a world frame and a first structure movement of the proximal repositionable structure relative to the base frame, wherein performing the first base movement and the first structure movement together moves a reference feature relative to the control feature, and wherein the reference feature is fixed relative to a portion of the proximal repositionable structure, and
commanding the actuator system to move the computer-assisted system based on the first base movement and the first structure movement.
